(19) United States
(12) Patent Application Publication
(10) Pub. No.: US 2025/0222161 A1
(43) Pub. Date: Jul. 10, 2025

(54) FUNCTIONALIZED POLYMER PARTICLES OF FIBERS FOR ODOR CONTROL IN WOUND CARE

(71) Applicant: Mölnycke Health Care AB, Göteborg (SE)

(72) Inventors: Milja Elias, Skepplanda, Ale (SE); Eric Wellner, Göteborg (SE)

(21) Appl. No.: 18/700,289

(22) PCT Filed: Oct. 11, 2022
(86) PCT No.: PCT/EP2022/078253

§ 371 Date: Apr. 11, 2024

(30) Foreign Application Priority Data
Oct. 15, 2021 (EP) 21202928.4

(51) Int. Cl. A61L 15/46; A61L 15/24; A61L 15/60; C08F 8/32; C08F 8/34; D06M 11/30; D06M 13/438; D06M 101/26

(52) U.S. Cl. CPC A61L 15/46; A61L 15/24; A61L 15/60; C08F 8/32; C08F 8/34; D06M 11/30; D06M 13/438; A61L 2300/202; D06M 2101/26

(57) ABSTRACT

Described are polymer particles or polymer fibers covalently bonded to N-chloroamines, N,N-dichloroamines, N-chloro sulfonamides or N,N-dichloro sulfonamides, for removing volatile organic compounds (VOCs) from a space above a wound. The removal of said VOCs is believed to be primarily or predominantly by chemical reaction of the VOCs with the N-chloro or the N,N dichloro group as covalently attached to the polymer. In particular, the functionalized polymer particles or polymer fibers are part of a wound dressing and have the functionality to control, in particular reduce, odor emanating from wounds, without interacting with the wound. The described dressings can be advantageously used, in particular, in the treatment of chronic wounds or infected wounds.

### FIELD OF THE INVENTION

The present invention relates to polymer particles or polymer fibers covalently bonded to N-chloroamines, N,N-dichloroamines, N-chloro sulfonamides or N,N-dichloro sulfonamides, for removing volatile organic compounds (VOCs) from a space above a wound, wherein the removal of said VOCs is primarily or predominantly by chemical reaction of the VOCs with the chloro-group as immobilized on the polymer.

In particular, the “active chlorine”-functionalized polymer particles or polymer fibers are part of a wound dressing and have the functionality to control, in particular reduce, odor emanating from wounds, without interacting with the wound, in particular without significantly or noticeably leaching into the wound. The dressings of the present invention can be advantageously used, in particular, in the treatment of chronic wounds or infected wounds.

In other aspects, the present invention relates to a method of controlling the odor above a wound space, said method including a step of providing the functionalized polymer particles or polymer fibers of the present invention.

### BACKGROUND OF THE INVENTION

As disclosed, for example, in the review article “A Comprehensive Review of Topical Odor-Controlling Treatment Options for Chronic Wounds” by A. Akhmetova et al., J Wound Ostomy Continence Nurs. (2016), 43(6), pages 598 to 609, various methods or products are available to manage wound odor. One of the more commonly used compositions for odor control over wounds is activated charcoal (which may be part of wound dressing). Charcoal is generally understood to absorb or “trap” VOCs due to the large active surface area of (activated) charcoal. No chemical reaction is believed to be involved in this physical adsorption mechanism.

In other approaches, silver or iodine are used as components in wound dressings, for example as coatings on a non-woven material or as impregnated in viscose rayon. Silver is believed to indirectly reduce odor by way of its antimicrobial properties. In that sense, silver should rather be seen as an antimicrobial agent (and as such may be part of the wound dressing of the present invention) than an odor reducing agent. Similarly, iodine is used to reduce the bacterial burden in the wound bed and therefore also reduces odor by way of disrupting bacterial wall membranes.

In an alternative approach, metronidazole (which is commonly used as a topically applied antibiotic) is used for the management of wound odor because of its ability to reduce odor producing anaerobic pathogens in selected wounds.

Natural compounds are also known as means to control wound malodor, for example honey or essential oils.

None of these approaches (silver, iodine, antibiotics, “natural” antibacterial products) are based on a mechanism of chemically reacting with the VOCs produced by bacteria/microbes/germs.

While all the known approaches as described above have their advantages, they are also associated with certain limitations. For example, the problem with wound control measures that involve physical adsorption, in particular charcoal, is the potential of saturation as the VOCs are not converted into non-odorous substances but are “stored” until capacity is reached. Furthermore, indirect methods that reduce or kill bacteria are not suitable for all wound types and only deal with odor due to bacterial infection. Such methods may not be suitable for all wounds, in particular not for chronic wounds.

Furthermore, many of these known approaches require that the (antimicrobial) agent penetrates into the wound in order to be effective, i.e. the agent is indeed an active ingredient that requires full clinical studies and certification.

Other sources of volatile organic compounds are metabolites produced by humans and secreted via the skin. Also, necrotic tissue as commonly present in chronic wounds may release VOCs.

A known approach to address odors in hygiene articles (e.g. in diapers) is the use of sulfonamides. As an example, U.S. Pat. No. 8,425,890 describes halo active aromatic sulfonamides for use in controlling odor in bodily fluids in personal hygiene articles such as diapers and sanitary napkins. According to the prior art, the sulfonamides, for example the halo active aromatic sulfonamides according to U.S. Pat. No. 8,425,890 are used as such, i.e. are used as chemical compounds that are added (e.g. coated onto) the overall product. For example, in U.S. Pat. No. 8,425,890, wood fluff is treated with an aqueous solution of the sulfonamide compounds.

Simply spraying chemical compounds onto a substrate (and then enclosing the substrate inside the article) may be a suitable approach for hygiene articles that are meant to take up bodily fluids secreted from the human body. However, the situation is different for wound dressings since wound dressings come in immediate contact with wound beds typically comprising open or compromised blood vessels, which means that the entry of chemically active compounds into the wound is generally not desirable or may even be detrimental to wound healing. Therefore, wound dressings that comprise active compounds that may leach out of the dressing are not desirable and the concept disclosed in U.S. Pat. No. 8,425,890 cannot be readily transferred to wound dressings.

### SUMMARY OF THE INVENTION

Overall, in view of the above, one object of the present invention is to provide means for controlling odor, in particular odor above wounds, wherein said means avoid or mitigate any or all of the disadvantages mentioned above.

In particular, an object of the present invention is to provide odor control means for wounds, in particular for chronic wounds, that do not expose the wound to direct contact with reactive chemical compounds and that permanently remove volatile organic compounds irrespective of their source.

This problem and others is/are solved, in a first aspect, by polymer particles or polymer fibers that are covalently bonded to N-chloroamines, or N,N-dichloroamines or covalently bonded to N-chloro or N,N-dichloro sulfonamides, wherein said functionalized polymer fibers or particles are used for removing volatile organic compounds, wherein the moiety as bonded to the polymer particles or polymer fibers is of a structure selected from the following:

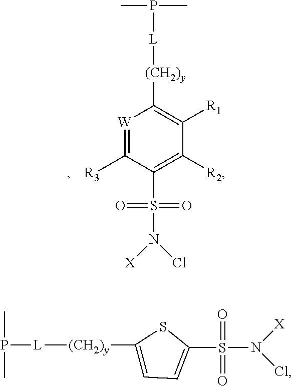

P-L-(CH₂)_{y}—SO₂—NXCl or P-L-(CH₂)_{y}—NXCl,
wherein:
   P- is a polymer;
   L is a linker;
   y is an integer greater than zero;
   W is N or —CH;
   R₁, R₂ and R₃ are independently selected from —H—, -alkyl, or -halogen;
   X is Na, H or Cl.

Without wishing to be bound by theory, the chlorine group of the moiety that is covalently bonded to the polymer is believed to remove VOCs primarily or predominantly by chemical reaction, i.e. the VOCs are cleaved into non (or less) odorous reaction products.

In embodiments, one of the following, preferably all of the following applies:
L-(CH₂)_{y} is —NH—(CH₂)_{y}; and/or
y is from 1 to 20, preferably from 1 to 12 or from 1 to 6 and/or
R₁ is selected from —H—, —CH₃—, or —Cl—; and/or
halogen is F, Cl or Br and/or:
R₂ and R₃ are —H—.

In embodiments particularly relating to the claimed N-chloro sulfonamides or N,N-dichloro sulfonamides, y is from 1 to 3

In embodiments particularly relating to the claimed N-chloroamines or N,N-dichloroamines, y is from 2 to 12

In embodiments, the polymer of the polymer particles or polymer fibers is based on polyacrylic acid, partially hydrolyzed acetyl-poly(vinylalcohol) or poly(vinyl alcohol), optionally a cross-linked polymer network of polyacrylic acid, partially hydrolyzed acetyl-poly(vinylalcohol) or poly(vinyl alcohol), or any copolymer or mixture thereof, wherein polyacrylic acids are preferred.

In embodiments, at least 0.1% of the alcohol or carboxylic acid groups originally present in the polymer are functionalized with a N-chloro or N,N-dichloro unit, preferably at least 0.5%.

In embodiments, the fraction of modified of polymer, i.e. degree of functionalization of the alcohol or carboxylic acid groups originally present in the polymer with a N-chloro or N,N-dichloro unit, is from 0.1% to 80%, preferably from 0.5% to 50%.

In embodiments, the amount of active chlorine as determined by the method provided in the experimental section below is from 0.1% to 20% (w/w), optionally from 0.3% to 16% (w/w).

In embodiments, the polymer particles are superabsorbent polymer particles (SAPs).

In accordance with the present invention and in accordance with the IUPAC definition provided in Pure Appl. Chem., Vol. 76, No. 4, pp. 889-906, 2004, a superabsorbent polymer is a polymer that can absorb and retain extremely large amounts of liquid relative to its own mass, preferably 10 times its weight or 100 times its weight.

In embodiments, the polymer fibers are superabsorbent fibers (SAFs).

In embodiments the median D⁵⁰ particle diameter of the polymer particles is from 80 μm to 600 μm, optionally from 150 μm to 400 μm; or wherein the average diameter of the fibers is from 1 μm to 300 μm, optionally from 5 μm to 100 μm.

In a second aspect, the above problem is/the above problems are solved by a wound dressing comprising the polymer particles or polymer fiber as described herein.

In embodiments, the fibers according to the present invention are preferentially incorporated in an airlaid fiber structure while the particles according to the present invention are preferentially incorporated in a foam.

In embodiments, at least 5% by weight, relative to the overall weight, optionally at least 10% by weight of the wound dressing is provided by the polymer particles or polymer fiber as described herein.

In embodiments, the polymer particles or polymer fibers as described herein have a grammage of at least 15 g/m², optionally at least 30 g/m² of the overall wound dressing.

In embodiments, the wound dressing furthermore comprises at least one of the following: (a) a backing layer; (b) at least one absorbent layer; (c) a wound contact layer.

In embodiments, said wound contact layer comprises or consists of a silicone gel.

In embodiments, the polymer particles or polymer fibers according to the present invention are provided as part of the absorbent layer.

In embodiments, the absorbent layer comprises superabsorbent particles and/or superabsorbent fibers not functionalized with N-chloro or N,N-dichloro groups, i.e. SAPs and/or SAFs, the primary function of which is to absorb wound exudate.

In embodiments, the wound dressing comprises superabsorbent particles and/or superabsorbent fibers not functionalized with N-chloro or N,N-dichloro sulfonamides. Wherein, in addition, polymer particles or polymer fibers functionalized with N-chloro or N,N-dichloro groups are provided separately from said superabsorbent particles or superabsorbent fibers not functionalized with N-chloro or N,N-dichloro groups.

In embodiments, the polymer particles or polymer fibers functionalized with N-chloro or N,N-dichloro groups are provided in a layer farther away from the area of the wound dressing that is intended to be put in contact with the wound (wound contact layer) relative to the layer that comprises the superabsorbent particles or superabsorbent fibers not functionalized with N-chloro or N,N-dichloro groups. This spatial separation ensures that those SAPs or SAFs that have the primary functionality of absorbing wound exudate are indeed close to the wound, while those SAPs and SAFs that are functionalized to cleave VOCs are removed away from the wound.

In a third aspect, the above-mentioned problem(s) is/are solved by a method of controlling odor above a wound space, said method comprising at least the following steps:
(a) providing polymer particles or polymer fibers as described above and herein in a wound dressing;
(b) bringing at least a fraction of said polymer particles or polymer fibers into contact with at least one volatile organic compound that is exuded from a wound and/or from the human skin in the vicinity of a wound;
(c) chemically reacting said volatile organic compound with N-chloro or N,N-dichloro groups as covalently bonded to said polymer particles or polymer fibers thus at least partially reducing odor emanating from a wound or from the area around a wound.

In embodiments, said at least one volatile organic compound is selected from heteroarylic, arylic compounds, sulfides, di-sulfides, trisulfides, ketones, alcohols, aldehydes, amines or carboxylic acids and esters.

In embodiments, said at least one volatile organic compound is selected from electron rich heteroarylic and arylic compounds.

In embodiments, said at least one volatile organic compound is selected from indole, 3-methyl indole, pyrimidine, acetophenone, isovaleric acid, 2,5-dimethylpyrazine, pyrrole, p-cresol, 2-aminoacetophenone, 6-methyl-5-heptene-2-one, cadaverine.

In embodiments, the wound is selected from chronic wounds (in contrast to “acute” wounds) and/or infected wounds.

### BRIEF DESCRIPTION OF THE FIGURES

FIG. 1 shows a synthesis scheme for functionalizing polymer particles or fibers with mono- or dichloro sulfonamide groups (1) and with mono or dichloro-amine groups (2). The synthesis is described in more detail below in the Experimental Section.

In this figure, EDC-HCl is N-(3-Dimethylaminopropyl)-N′-ethylcarbodiimide hydrochloride, NHS is N-Hydroxy-succinimide, MES is 4-Morpholineethanesulfonic acid monohydrate, HCl is Hydrochloric acid, NaHCO₃ is Sodium bicarbonate, NaOCl is sodium hypochlorite and HAc is Acetic acid.

FIG. 2 shows SEM images of A) non-functionalized superabsorbent fibers (SAFs) B) N-chloro-sulfonamide modified SAF and C) N,N-dichloro-sulfonamide modified SAF.

As can be taken from the scanning electron microscopy pictures, the diameter of unmodified SAF is around 30 μm whereas the diameter of N-chloro-sulfonamide modified SAF is around 40 μm and that of N,N-dichloro-sulfonamide modified SAF around 50 μm.

FIG. 3 shows that superabsorbent particles (here: polyacrylic acid, these particles are also referred to as “PAA”) and superabsorbent fibers SAF (also based on polyacrylic acid) as such, i.e. without being modified in accordance with the present invention essentially do not affect (reduce) the amount of volatile organic compounds. In these Figures and in the following Figures the VOCs are: Indole, 3-Methyl indole (3Melnd), Pyrimidine (Pyrim), Acetophenone (APh), Isovaleric acid (Isoval), 2,5-Dimethylpyrazine (2,5-Dimepy), Pyrrole, p-Cresol, 2-aminoacetophenone (2-AAph), 6-Methyl-5-heptene-2-one (6M-hept-one) and Cadaverine (CAD). The one exception to the general observation that the unmodified polymers do not reduce the amount of VOCs is that superabsorbent fibers as such indeed do reduce (or even remove) cadaverine (see lower panel of FIG. 3).

FIGS. 4 to 7 show how functionalization of SAPs and SAFs with mono- or dichloro sulfonamide groups or with mono or dichloro-amine groups leads to a significant to essentially complete removal of a large range of VOCs.

FIG. 4, upper panel, shows the effect of functionalized SAF polymer 2 as described in the experimental section below on selected VOCs while FIG. 4, lower panel, shows the effect of functionalized SAP Polymer 1 as described in the experimental section below on selected VOCs

FIG. 5, upper panel, shows the effect of different amounts of functionalized SAP Polymer 2 as described in the experimental section below on selected VOCs while FIG. 5, lower panel, shows the effect of functionalized SAF Polymer 3 as described in the experimental section below on selected VOCs

FIG. 6, upper panel, shows the effect of functionalized SAF polymer 5 as described in the experimental section below on selected VOCs while FIG. 6, lower panel, shows the effect of functionalized SAF Polymer 4 as described in the experimental section below on selected VOCs

FIG. 7, upper panel, shows the effect of functionalized SAF polymer 6 as described in the experimental section below on selected VOCs while FIG. 7, lower panel, shows the effect of functionalized SAF Polymer 7 as described in the experimental section below on selected VOCs

FIGS. 8 and 9 show tables providing values for the % reduction of VOCs for functionalized polymers as described in the Experimental Section below.

### DETAILED DESCRIPTION OF EMBODIMENTS OF THE INVENTION

One advantage of the present invention is that the volatile organic compounds are not simply absorbed but that an actual chemical reaction takes place that converts the odorous VOCs into non-odorous or otherwise harmless smaller molecules. Since a chemical reaction takes place, there is, in principle, no limitation to the amount of VOCs that can be removed from a wound space or the vicinity of a wound space by the functionalized polymer particles or fibers as described herein.

Also, importantly, due to immobilization of the chemically reactive moiety with the N-chloro or the N,N dichloro group to a polymer backbone, release of a water soluble active component into the wound is avoided or at least substantially avoided.

This puts the present invention in contrast to existing odor control measures in hygiene articles, wherein sulfonamides are not truly immobilized or not at all immobilized, for example merely sprayed or coated onto a substrate and thus may be easily released into a fluid system and thus may leach into the wound, which is generally undesirable (see “Background” section)

A further advantage of the present invention is that polymer particles or polymer fibers that are already commonly used in wound dressings, for example superabsorbent polymers or superabsorbent fibers can be functionalized to carry the herein disclosed N-chloro or the N,N dichloro group

In embodiments, superabsorbent polymer particles or superabsorbent fibers are partially functionalized with the N-chloro or the N,N dichloro group as disclosed herein thus leading to superabsorbent particles or superabsorbent fibers that are still superabsorbent but that also have VOCs cleaving functionality.

In accordance with the present invention “partially functionalized” means that 5% to 75% of all available OH-groups of a particle or fiber are functionalized, preferably 15% to 60%.

In other embodiments, polymer fibers or polymer particles are fully or essentially fully functionalized with N-chloro or the N,N dichloro groups as disclosed herein and such fully or essentially fully functionalized superabsorbent fibers or superabsorbent particles are used together with the same or different superabsorbent polymer particles or superabsorbent polymer fibers that are not functionalized or are only partially functionalized and thus primarily function as superabsorbent materials.

In other embodiments, several layers are provided in a wound dressing, wherein the layer closer to the wound space comprises superabsorbent particles or superabsorbent fibers that are not functionalized or only partially functionalized with N-chloro or N,N dichloro groups while a second, separate layer of superabsorbent particles or superabsorbent fibers is provided farther away from the wound space, which then comprises fully essentially fully functionalized superabsorbent polymers or superabsorbent fibers having the primary functionality to cleave VOCs.

A further advantage of the present invention is that due to the mechanism of chemically reacting with VOCs, i.e. directly removing the VOCs, the mechanism that leads to VOCs or the origin of the VOCs is irrelevant and wounds can be treated in which the origin of the VOCs is not primarily of bacterial origin.

An additional benefit of the sulfonamide functionalized polymer particles or polymer fibers is that in use, i.e. in contact with wound exudate, the claimed and disclosed N-chloro or N,N dichloro group are capable of releasing hypochlorite which is known to have antimicrobial properties and can thus also contribute to the lowering of the bioburden in a dressing and/or in the wound.

### EXAMPLES (EXPERIMENTAL)

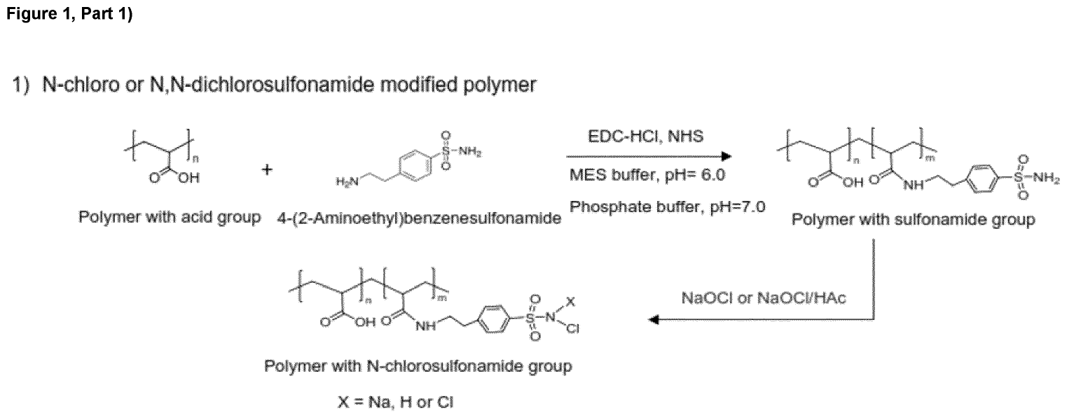

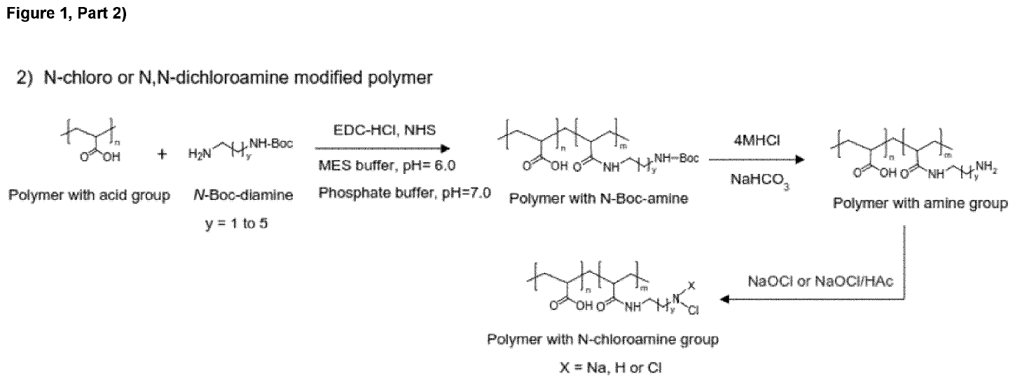

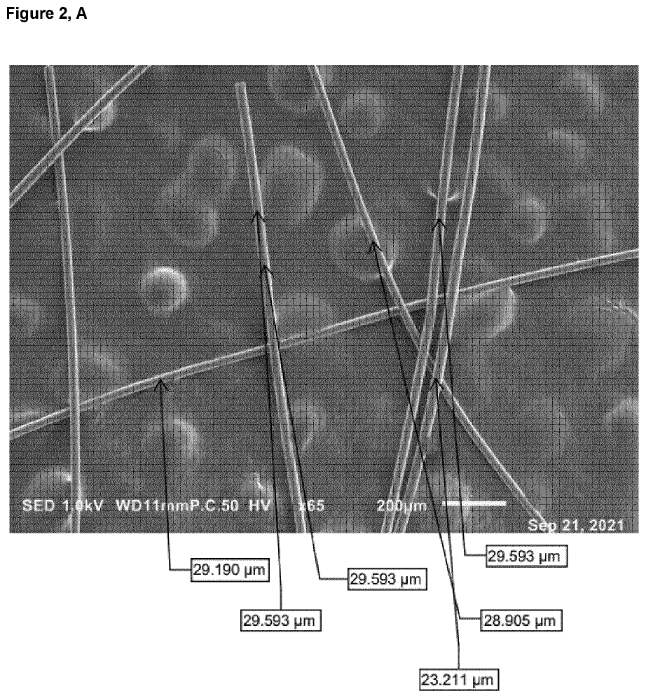

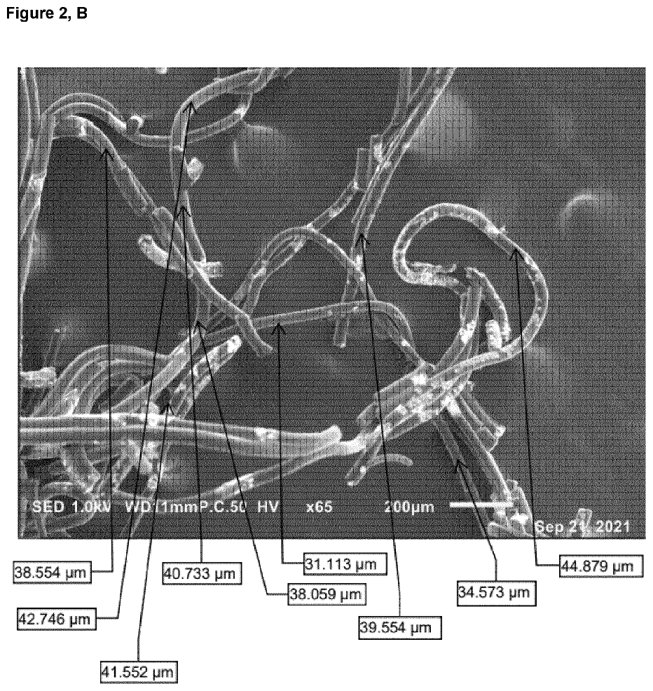

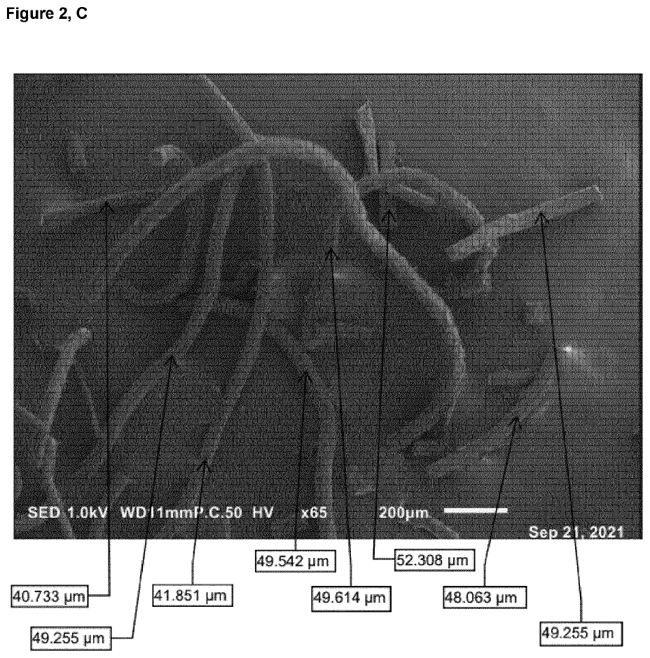

### 1.) Synthesis (See FIG. 1)

### Reagents:

Polyacrylic acid (PAA)—Mol. Wt. —1,250,000 Da—Sigma Aldrich
SAF—Super absorbent fiber—Technical Absorbents
AEBSA—4—(2-Aminoethyl)benzenesulfonamide, 99%—Thermo Fisher Scientific
MES—4-Morpholineethanesulfonic acid monohydrate—Thermo Scientific™ BupH MES Buffered Saline Pack
NaOCl—sodium hypochlorite (6-14% active chlorine)—Honeywell Fluka
HAc—glacial acetic acid—Sigma Aldrich
N-Boc-ethylenediamine—Sigma Aldrich
N-Boc-1,6-hexanediamine—Sigma Aldrich

### Synthesis of Polyacrylic Acid Polymer Particles Functionalized with N-Chloro/N,N-Dichloro Sulfonamide

### Step 1—EDC coupling

1 g PAA was added to 65 mL MES buffer (0.05M, pH=6.0) in a 250 mL RB flask. Then 0.760 g EDC-HCl and 0.920 g NHS dissolved in 15 mL MES buffer (0.05M, pH=6.0) was added. This was stirred at RT for 30 minutes. Then 1.2 g AEBSA dissolved in 20 mL phosphate buffer solution (0.1M, pH=7.0) was added and the reaction continued at RT for 4 h. After 4 h, the sulfonamide bound polymer was washed with deionized water to remove all the unreacted reagents and impurities. The formed product was then dried at RT.

### Step 2—Chlorination

Polymer 1-1 g of the sulfonamide bound polymer was taken in a round bottom flask and added deionized water to swell the polymer. Then 16 mL of NaOCl solution and 1 mL HAc was added. The reaction was continued in an ice bath for 4 h. The formed product was washed with large amount of deionised water till the washings did not show presence of chlorine (Absence of blue color with KI and starch solution).

### Synthesis of Polymer Fibers Functionalized with N-Chloro/N,N-Dichloro Sulfonamides

### Step 1—EDC Coupling

1 g SAF was added to a 250 mL RB flask followed by 20 mL of MES buffer. 0.76 g EDC and 0.92 g NHS was dissolved in 8 mL of MES buffer and added to SAF in MES buffer. After stirring for 30 minutes, 1.2 g AEBSA dissolved in 20 mL of phosphate buffer (pH=7.0) was added. Stirred for 4 h. The product was washed several times with deionised water and dried at room temperature.

### Step 2—Chlorination

Polymer 2—1 g of sulfonamide bound superabsorbent fibers (SAF) was added to 250 mL RB flask. Approx. 120 mL deionised water was added to swell the fiber. Then 16 mL NaOCl and 1 mL gl. acetic acid was added and the reaction continued for 4 h in an ice bath. The formed product was washed with large amount of deionised water till the washings did not show presence of chlorine (Absence of blue color with KI and starch solution). Dried at room temperature.

Polymer 3—1 g of sulfonamide bound SAF was added to 250 mL RB flask. Approx. 120 mL deionised water was added to swell the fiber. Then 16 mL NaOCl was added and the reaction continued for 4 h in an ice bath. The formed product was washed with large amount of deionised water till the washings did not show presence of chlorine (Absence of blue color with KI and starch solution). Dried at room temperature.

### Synthesis of Polymer Fibers Functionalized with N-Chloro/N,N-Dichloroamines

### Step 1—EDC Coupling was Carried Out as Mentioned Above for SAF with N-Boc Protected Diamines Instead of AEBSA

### Step 2—Deprotection

−0.25 g of fibers modified with N-Boc protected amine was added to 10 mL 4MHCl and stirred at room temperature for 3 h. This was filtered, added deionised water and neutralised with sodium bicarbonate. The formed amine modified fibers were filtered, washed with deionized water and dried at RT

### Step 3—Chlorination

Polymer 4—Same procedure was used as for polymer 2 to get N,N-dichloro-ethylene diamine modified SAF.

Polymer 5—Same procedure was used as for polymer 3 to get N-chloro-ethylene diamine modified SAF.

Polymer 6—Same procedure as for polymer 2 to get N,N-dichloro-hexamethylene diamine modified SAF.

Polymer 7—Same procedure as for polymer 3 to get N-chloro-hexamethylene diamine modified SAF.

### 2.) Determination of Amount of Active Chlorine in the Modified Polymers-Iodometric Titration

0.5 g of KI was added to 10 mL deionized water. 50 mg of the modified polymer was added followed by 3 drops of 1% starch solution as indicator. A blue color formed which disappears at end point upon titration with 0.1 N (for N,N-dichloro compounds) or 0.01 N (for N-chloro compounds) sodium thiosulphate solution. Titration was done in triplicates for all samples.

The amount of active chlorine was calculated using the following equation:

$${Cl}^{+} ( \% ) = \frac{35.45 XNXV}{2 XW} \times 100$$

wherein N is the equivalent concentration (N) and V is the volume (L) of the sodium thiosulphate solution used for titration, W is the weight of the modified polymer (g).

The following amounts of active chlorine were determined using this titration method:
Polymer 1—Polyacrylic acid modified with N,N-dichlorosulfonamide ˜4%
Polymer 2—SAF modified with N,N-dichlorosulfonamide—˜4% and ˜14%
Polymer 3—SAF modified with N-chlorosulfonamide—˜2%
Polymer 4—SAF modified with N,N-dichloro-ethylene diamine ˜8%
Polymer 5—SAF modified with N-chloro-ethylene diamine—˜0.3-˜0.6%
Polymer 6—SAF modified with N,N-dichloro-diaminohexane—˜8%
Polymer 7—SAF modified with N-chloro-diaminohexane—˜1%
   3.) Testing Reduction of VOC Concentration with Modified Polymers

VOCs emitted by bacteria commonly found in wound (P. aeruginosa, S. aureus, E. coli) were selected from literature. These were divided into two mixtures for ease of calibration, because of co-eluting peaks, to get good peak separation.
Mix 1:—Indole, 3-Methyl indole (3Melnd), Pyrimidine (Pyrim), Acetophenone (APh), Isovaleric acid (Isoval), 2,5-Dimethylpyrazine (2,5-Dimepy)
Mix 2:—Pyrrole, p-Cresol, 2-aminoacetophenone (2-AAph), 6-Methyl-5-heptene-2-one (6M-hept-one), Cadaverine (CAD).

### Method:

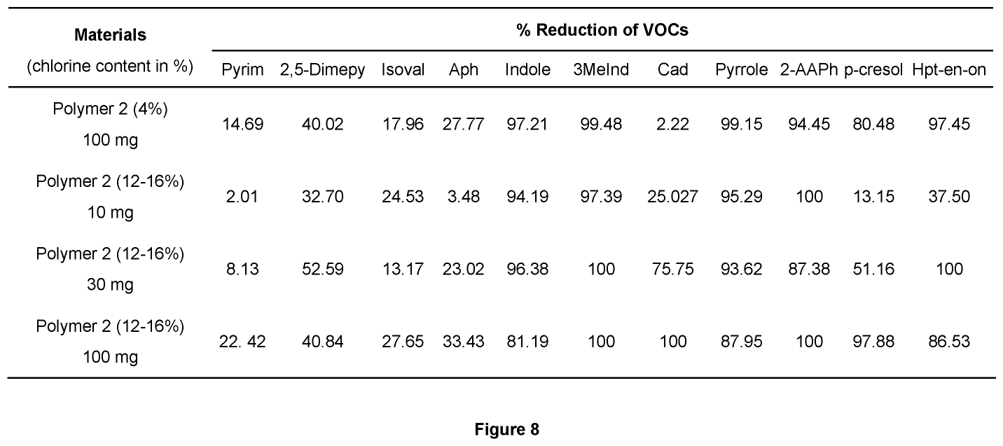

VOCs—A concentration in the validated range was chosen and the solutions were prepared as Mix 1 and Mix 2 in simulated wound fluid (SWF).
Polymer material (polymer 1 to 7, 100 mg) was added to 2.5 mL each of Mix1 and Mix 2 solution in SWF. For polymer 2 the amount was varied as specified in FIG. 8.
Vortexed for one minute.
Incubated with shaking at 35 deg C. for 24 h.
Supernatant analysed by HPLC-MS.

The concentration of VOCs in SWF after incubation for 24 h was measured and from that the percentage reduction after adding the materials was calculated.

### 4.) Results

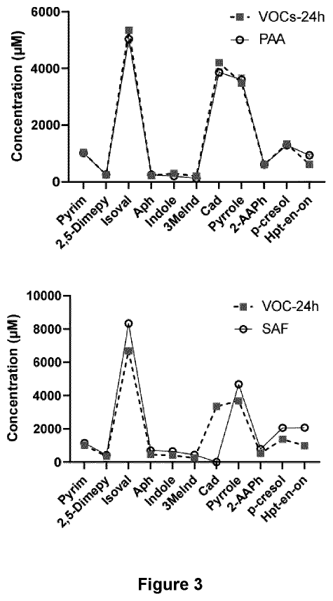

As shown in FIG. 3, the non-modified (non-functionalized) polymer particles or fibers do not affect the VOC concentration (with the exception of SAFs reducing cadaverine).

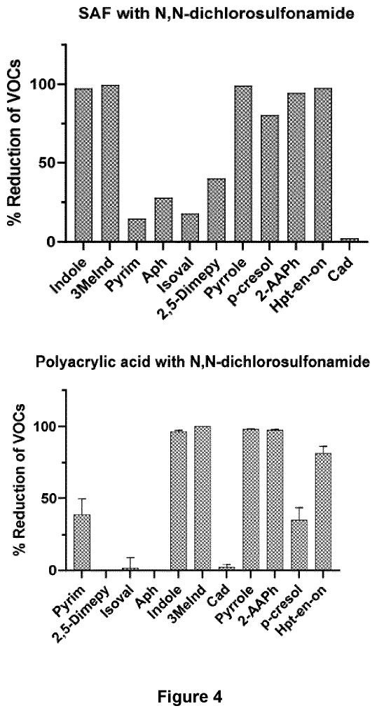

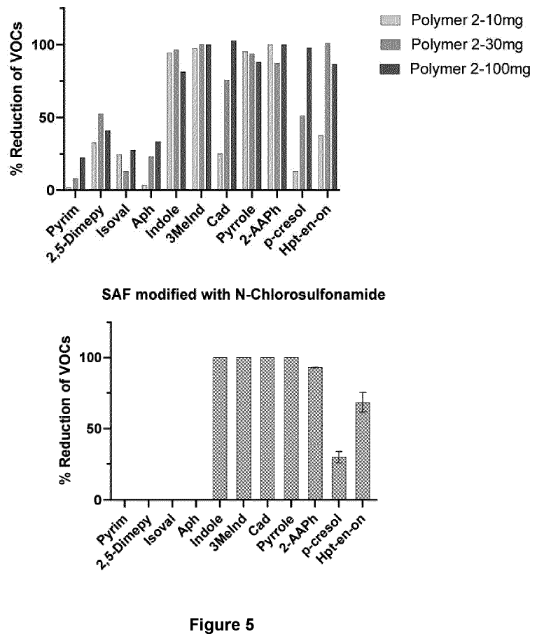

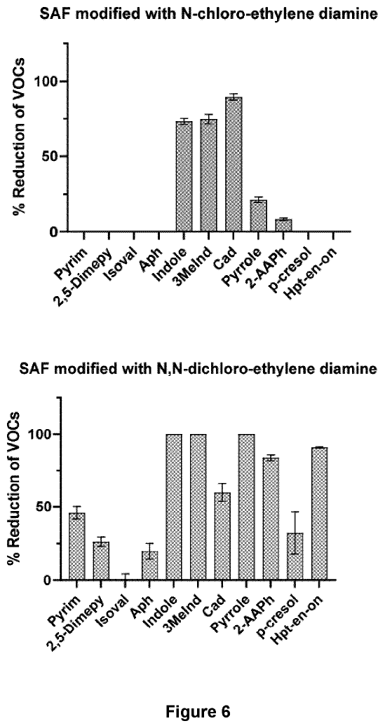

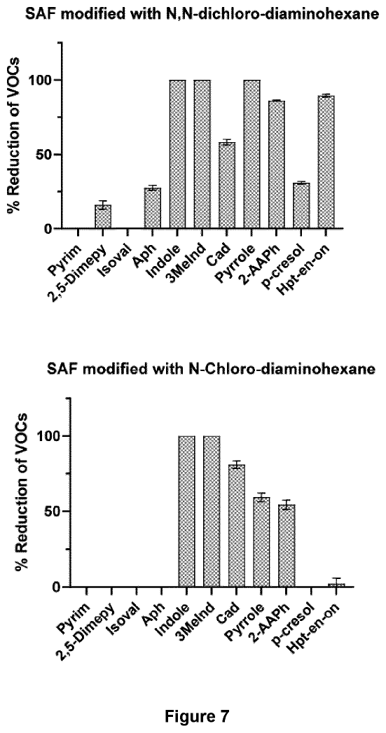

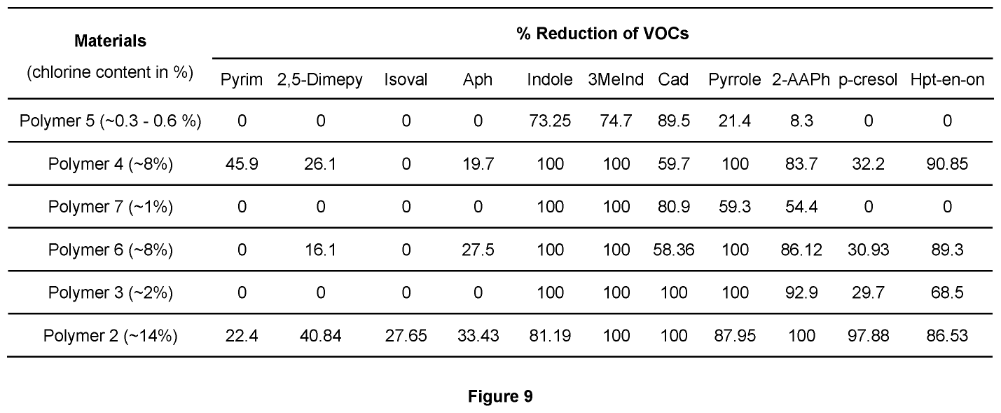

FIGS. 4 to 7 as well as the tables reproduced in FIG. 8 and FIG. 9 show that, depending on the specific modification (Polymers 1 to 7 as described above) essentially all of the exemplarily chosen VOCs can be removed by way of chemical reaction. FIG. 5 shows that at least VOCs can already be removed by a comparatively low amount of functionalized polymer.

FIGS. 4 to 9 provide evidence that the inventive concept works well for both polymer particles and fibers and that functionalization with chloroamines and with chlorosulfonamides is effective in reducing VOCs.

1. A polymer particle or a polymer fiber covalently bonded to a N-chloroamine or a N,N-dichloroamine or covalently bonded to a N-chlorosulfonamide or N,N-dichlorosulfonamide, for removing a volatile organic compound, wherein a moiety as bonded to the polymer particle or the polymer fiber is of a structure selected from the following: 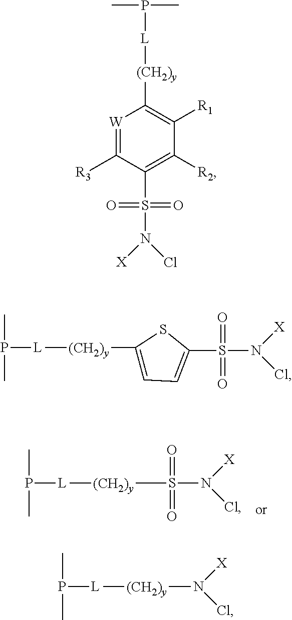
wherein:
P- is a polymer;
L is a linker;
y is an integer greater than zero,
W is N or —CH;
R₁, R₂ and R₃ are independently selected from —H—, -alkyl, or -halogen; and
X is Na, H or Cl.

2. The polymer particle or polymer fiber of claim 1, wherein
L-(CH₂)_{y} is —NH—(CH₂)_{y}; and/or
y is from 1 to 20; and/or
R₁ is selected from —H—, —CH₃—, or —Cl—; and/or
halogen is F, Cl or Br; and/or
R₂ and R₃ are —H—.

3. The polymer particle or polymer fiber of claim 1, wherein the polymer is based on polyacrylic acid, partially hydrolyzed acetyl-poly(vinylalcohol) or poly(vinyl alcohol), optionally a cross-linked polymer network of polyacrylic acid, partially hydrolyzed acetyl-poly(vinylalcohol) or poly(vinyl alcohol), or any copolymer or mixture thereof.

4. The polymer particle or polymer fiber according to claim 1, wherein at least 0.1% of the alcohol or carboxylic acid groups originally present in the polymer are functionalized with a N-chloro or N,N-dichloro unit.

5. The polymer particle or polymer fiber according to claim 1, wherein the amount of active chlorine as determined by the method provided in the description is from 0.1% to 20% (w/w).

6. The polymer particle or polymer fiber according to claim 1, wherein the median D50 particle diameter of the polymer particle is from 80 μm to 600 μm; or wherein the average diameter of the fiber is from 1 μm to 300 μm.

7. A wound dressing, comprising the polymer particle or polymer fiber of claim 1.

8. The wound dressing according to claim 7, comprising at least 5% by weight of said polymer particle or polymer fiber, relative to the overall weight of the dressing, and/or wherein the polymer particle or polymer fiber have a grammage of at least 15 g/m², of the overall wound dressing.

9. The wound dressing according to claim 7, furthermore comprising at least one of the following:
(a) a backing layer;
(b) at least one absorbent layer; and
(c) a wound contact layer, said wound contact layer optionally comprising a silicone gel.

10. The wound dressing according to claim 8, wherein the polymer particle or polymer fiber are provided as part of the at least one absorbent layer, optionally wherein the absorbent layer comprises a superabsorbent particle or superabsorbent fiber not functionalized with a N-chloro or N,N-dichloro group.

11. The wound dressing according to claim 7, wherein the wound dressing also comprises a superabsorbent or superabsorbent fibers not functionalized with a N-chloro or N,N-dichloro sulfonamide; wherein the polymer particle or polymer fiber functionalized with a N-chloro or N,N-dichloro sulfonamide are provided separately from the superabsorbent or superabsorbent not functionalized with a N-chloro or N,N-dichloro sulfonamides.

12. A method of controlling odor above a wound space, said method comprising the following steps:
(a) providing a polymer particle or a polymer fiber according to claim 1 in a wound dressing;
(b) bringing at least a fraction of said polymer particle or polymer fiber into contact with at least one volatile organic compound that is exuded from a wound and/or from the human skin in the vicinity of a wound; and
(c) chemically reacting said volatile organic compound with the N-chloro or N,N-dichloro group as covalently bonded to said polymer particle or polymer fiber thus at least partially reducing odor emanating from a wound or from the area around a wound.

13. The method according to claim 12, wherein said at least one volatile organic compound is selected from the group consisting of a heteroarylic, arylic compounds, sulfides, di-sulfides, trisulfides, ketones, alcohols, aldehydes, amines, carboxylic acids, and esters.

14. The method according to claim 12, wherein said at least one volatile organic compound is selected from the group consisting of indole, 3-methyl indole, pyrimidine, acetophenone, isovaleric acid, 2,5-dimethylpyrazine, pyrrole, p-cresol, 2-aminoacetophenone, 6-methyl-5-heptene-2-one, and cadaverine, or a combination thereof.

15. The method according to claim 12, wherein the wound is selected from a chronic wound and/or an infected wound.

16. The wound dressing according to claim 11, wherein the polymer particle or polymer fiber functionalized with a N-chlorosulfonamide or N,N-dichlorosulfonamide is provided in a layer farther away from the area of the wound dressing that is intended to be put in contact with the wound than a layer that comprises the superabsorbent particle or superabsorbent fiber not functionalized with a N-chlorosulfonamide or N,N-dichlorosulfonamide.

17. The polymer particle or polymer fiber of claim 1, wherein the structure is

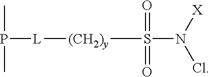

18. The polymer particle or polymer fiber of claim 1, wherein the structure is

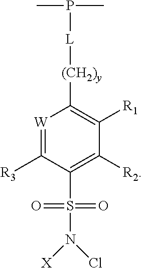

19. The polymer particle or polymer fiber of claim 1, wherein the structure is

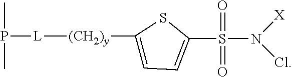

20. The polymer particle or polymer fiber of claim 1, wherein the structure is [chemical structure]
